(19) United States
(12) Patent Application Publication
(10) Pub. No.: US 2006/0163067 A1
(43) Pub. Date: Jul. 27, 2006

(54) Apparatus for the manufacture of a disposable electrophoresis cassette and method thereof

(76) Inventors: Pierre Sevigny, Montreal (CA); Roy Dominique, St-Lazare (CA)

Correspondence Address: DAVID S. RESNICK, 100 SUMMER STREET, NIXON PEABODY LLP, BOSTON, MA, 02110-2131, US

(21) Appl. No.: 10/506,723

(22) PCT Filed: Mar. 6, 2003
(86) PCT No.: PCT/CA03/00312

§ 371 Date: Jul. 25, 2005

(30) Foreign Application Priority Data
Mar. 7, 2002 (US) 10091549

(51) Int. Cl. C07K 1/26; G01N 27/447

(52) U.S. Cl. 204/465; 204/615

(57) ABSTRACT

The present invention relates to a mold for the manufacture of an electrophoresis cassette (10), the mold comprising a body having a cassette molding part formed on one face thereof, the cassette molding part being surrounded by a peripheral sheet engaging portion extending in a plane located at a different elevation than the cassette molding part to provide for substantially uniform stretching of the sheet on the cassette molding part. The present invention also relates to a molding method for the manufacture of an electrophoresis cassette, a method for filing an electrophoresis medium into an electrophoresis cassette and to a comb (22) for an electrophoresis cassette adapted to be removably inserted into the cassette comprising at least one tooth (32) having protrusion (104) provided thereto for preventing the electrophoresis medium gel attachment to the comb.

### BACKGROUND OF THE INVENTION

(a) Field of the Invention

The present invention is related to an apparatus for the manufacture of a disposable electrophoresis cassette and to the manufacture process thereof.

(b) Description of Prior Art

Electrophoresis is a well known separation technique that requires the application of electrical current at both poles of a cassette or plate to force samples through an electrophoretic medium that acts as a molecular sieve. The application of a difference of potential between the upper section and the lower section of the cassette assumes the creation of two areas sealed from each other. Because current is transmitted via two separate buffer reservoirs, it is necessary to apply a pressure or force on the cassette so that the seals properly operate. It is therefore imperative that the whole system, including the cassette, possess some rigidity.

Conventional electrophoresis cassettes are made of two glass plates spaced apart with plastic spacers or tongues (often in plastic, ABS, rubber or other non-conductive material) to create a space therebetween while ensuring that the sides of the assembly are properly sealed. Importantly, the spacers must not conduct electrical current. The assembly is generally maintained together with clamps, and it is often necessary to reinforce the seals with hot agar or grease (like petroleum jelly). When the gel is cast into the cassette, a comb element is introduced at one end of the assembly (usually define as the Top of the cassette) to create one or more reservoirs or wells thereafter wherein the sample(s) will be received later. The shape of the comb may comprise various numbers and sizes of reservoirs, depending on the application required and the size of the cassette. For example, a preparation gel necessitate less reservoirs, while an analytical gel will require more reservoirs and the width thereof will depend on the resolution desired.

However, such assemblies have several drawbacks and limitations. The assembling operation requires dexterity and is a time-consuming operation, because it is done manually. The plates are conventionally made of glass, and thus must be handled with care. Further, they must be carefully cleaned to obtain good results. Finally, manipulation of acrylamide gel, a commonly used electrophoretic medium, represents a long-term danger for the health of operators since such gel is highly toxic.

More recently, to simplify the assembling work of operators and reduce poisoning and manipulation hazards, pre-cast cassettes already containing the gel have been made available commercially. The cassettes comprise an acrylamide gel, and a comb is provided at one extremity thereof. However, the cost of these cassettes is prohibitive, and demolding thereof, for visualization of the results, is a delicate and complicated procedure. In addition, the comb is produced by injection molding, and is used to form the wells or reservoirs in the gel. They generally represent an important part of the total cost of the cassette.

To be economically feasible and capable of supporting, without substantial bending, the mechanical forces applied thereon, cassettes containing pre-cast electrophoresis medium, must be rigid enough and made of a material economically sound and preferably recyclable, such as for example thermoplastic materials like polymethylmethacrylate (PMMA). However, conventionally, in order to be sufficiently rigid, the plates must be relatively thick. Two obvious problems therefore become apparent: a) the amount of thermoplastic material required is significant, thus increasing the cost, which is not suitable for a disposable device; and b) maintaining the gel at an appropriate operating temperature is complicated, because the thick walls of the thermoplastic material act as a dielectric material. Thicker plastic walls also affect the diffusion of the heat generated during the electrophoretic process, creating temperature gradients within the electrophoresis medium, and non-uniform migration of the samples analyzed.

Conventional processes for filling the cassettes are generally standard, irrespective of the electrophoretic medium. Typically, a gel comprising a mixture of acrylamide and bis-acrylamide, a buffer like tris-borate ethylenediamine (EDTA), tris-acetate-EDTA, tris-glycine, tricine, and a polymerization initiator are injected or cast into the cassette. Some of these products are neurotoxic and/or irritant, and must therefore be handled with extreme care. A laboratory pipette or a pump can be used to fill the cassette from the top with the liquid medium. Once the cassette is filled, a comb closes the top of the cassette. The comb has a design such that it contains one or more teeth forming reservoirs in the gel wherein the sample will be placed later. After polymerization of the medium, the comb is removed, as well as a separator present in the lower portion of the cassette. The cassette is then placed in an electrophoresis apparatus wherein the lower and upper portions of the gel will be in contact with two independent buffer solutions relating to the electrodes. The samples are then introduced in the reservoirs, and current is applied to separate the various components of each sample. After completion of the separation, the medium is removed from the cassette for further processing, i.e., coloration, photograph and analysis.

Again, such system and procedure have various major drawbacks and limitations. As stated above, manual filling of the cassette requires great care and dexterity, not to mention exposure of the operator to toxic chemicals. Further, undesirable bubbles often form during filling, and installation of the comb after filling may also create bubbles at the bottom of the teeth. Such air bubbles must be avoided at all times, since they interfere significantly with the samples migrating in the polymerized gel during the electrophoresis procedure.

Pre-cast gels have been marketed recently, but have not been able to overcome other problems mentioned above for cassettes containing the same, such as prohibitive costs. One of the main reason is that the cassettes are obtained by injection molding, which is a costly and relatively slow process because of the significant amount of plastic required for injection, the cost of the plastic material itself, and the time necessary to allow complete cooling of the cassette thus obtained. In addition, because the cassettes are made of a thermoplastic material, gel polymerization is greatly affected and slowed down because the polymer absorbs free radicals generated by the chain reaction of the polymerization or free oxygen molecules which affect the polymerization efficiency. As a result, the polymerized electrophoretic medium does not “stick” do the cassette inner surfaces. An expensive coating layer or overlay must therefore be applied on the thermoplastic material surfaces to minimize this problem and ensure proper polymerization quality and speed.

The electrophoresis operation necessitates the application of a voltage across the gel that generates heat that must be somehow dissipated. During the heat dissipation process, if the temperature of the gel is not uniform, it causes distortion in the separated protein or polynucleic acid bands shown as a “smiling effect” or loss of resolution (thicker bands). Such heat is therefore a critical problem because it limits the rate at which gels can be run. Increasing temperatures reduces the resistance and increases current at a given voltage. Although the net effect is a shorter run, excessive temperature can lead to undesirable band broadening. It is therefore preferable to run at a higher voltage and a constant lower temperature.

It would be highly desirable to be provided with a cassette having thin plastic walls using a minimal amount of plastic, being adapted to any existing electrophoresis boxes and systems, being low-cost to produce and being easy to fill.

### SUMMARY OF THE INVENTION

One aim of the present invention is to provide a mold that allows easy preparation of a disposable electrophoresis cassette within the specification.

A further aim of the present invention is to provide a relatively simple and efficient manufacturing process for the production of a disposable electrophoresis cassette.

A still further aim of the present invention is to provide a mold and a process for industrial production in a large volume and at lower manufacturing cost of electrophoresis cassettes.

Another aim of the present invention is to provide a gel filing method allowing easy preparation of a disposable electrophoresis cassette.

Another aim of the present invention is to provide a comb for a disposable electrophoresis cassette that prevents gel polymerization between the comb and the cassette walls during utilization.

In accordance with the present invention there is provided a mold for the manufacture of an electrophoresis cassette, the mold comprising a body having a cassette molding part formed on one face thereof, the cassette molding part being surrounded by a peripheral sheet engaging portion extending in a plane located at a different elevation than the cassette molding part to provide for substantially uniform stretching of the sheet on the cassette molding part.

In accordance with the present invention, there is also provided a molding method for the manufacture of an electrophoresis cassette comprising the steps of:

a) heating a thermoforming material applied on a thermoforming mold suitable for the manufacture of an electrophoresis cassette;

b) applying a pressure on the material to closely maintain the material on the mold;

c) stretching the material to obtain an uniformly distributed material surface;

d) cooling the material to form a molded material surface with cooling parameters adapted to provide an uniformly distributed material surface; and

e) providing holes in a peripheral portion of the electrophoresis cassette facing a peripheral portion comprising reservoirs entries.

In accordance with the present invention, there is further provided a method for filing an electrophoresis medium into an electrophoresis cassette comprising the steps of:

a) sealing at least one aperture of the cassette;

b) injecting the electrophoresis medium into the cassette;

c) applying a pressure onto the cassette in order to generate a flat and even electrophoresis separation area in the electrophoresis medium.

In accordance with the present invention, there is still further provided a comb for an electrophoresis cassette adapted to be removably inserted into the cassette comprising at least one tooth having protrusion provided thereto for preventing the electrophoresis medium gel attachment to the comb and/or for preventing acrylamide polymerization.

### BRIEF DESCRIPTION OF THE DRAWINGS

FIG. 1 is a perspective exploded view of a cassette and the corresponding support plate in accordance with an embodiment of the present invention;

FIGS. 2A-2D are a partial perspective view of a comb developed in accordance with an embodiment of the present invention;

FIGS. 3A and 3B are partial front and rear perspective views of a hybrid comb developed in accordance with an embodiment of the present invention.

FIG. 4 is a partial view of the support plate developed for supporting the present cassette;

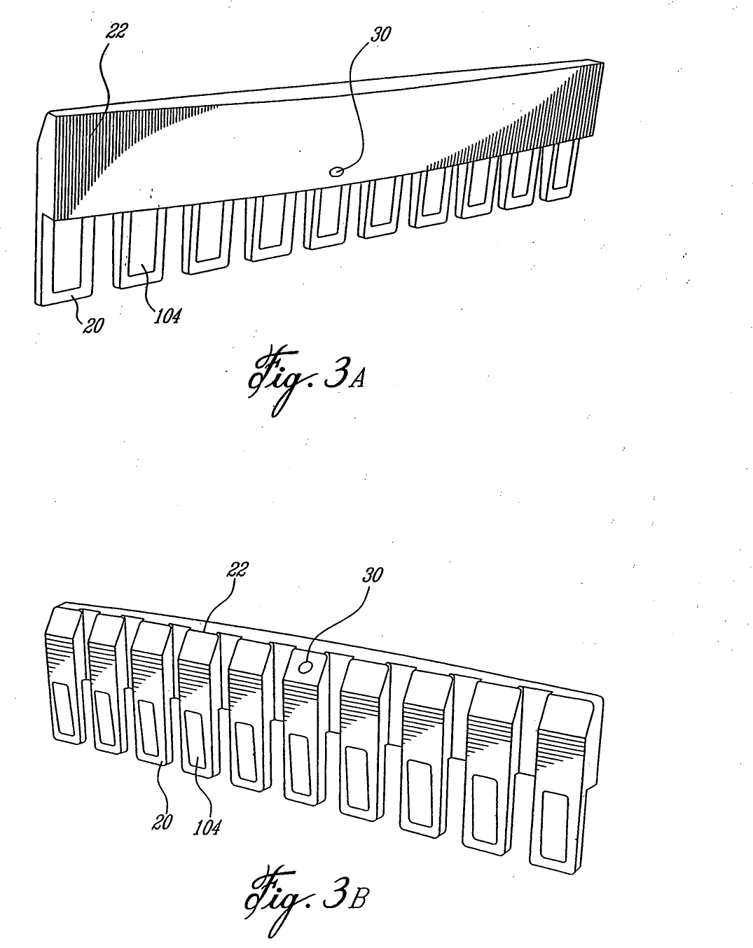

FIG. 5 is a perspective view of the mold used to prepare the cassette of FIG. 1;

FIGS. 6A and 6B are cross-sectional views of the mold used to prepare the cassette of FIG. 1; and

FIG. 7 is a side view of another embodiment of the mold of the present invention.

### DETAILED DESCRIPTION OF THE INVENTION

The invention relates to the field of electrophoresis, and more particularly to a cassette suitable therefor. It is to be assumed that the gel used as the electrophoretic medium is preferably an acrylamide (or polyacrylamide) gel, whether cross-linked or not. However, other conventional and well known electrophoretic media such as agarose gel or starch gel, can be used. Polyacrylamide gel is particularly preferred because it is transparent, electrically neutral, and can be made in various pore sizes. Other co-monomers well known in the field include N,N′-methylenebisacrylamide, N,N-bisacrylylcystamine, N,N′-(1,2-dihydroxyethylene)bisacrylamide, N,N′-diallyl-tartardiamide, and the like.

The cassette of the present invention is a cassette defined by a reservoir, made of plastic by a process allowing the formation of very thin surfaces, preferably thermoforming or “thin wall” molding, a cover made of a material suitable for the manufacture of an electrophoresis cassette and providing sufficient rigidity to have a structure allowing the cassette to stay flat and linear. A male mold type is used to provide more precision and allow molding pieces with smaller-narrower details while female molding provide pieces with larger details. The cassette also comprises a comb, made of molded plastic, designed to inject an electrophoretic medium and/or to mold wells into the cast gel in order to have cavities to receive the sample to be analyzed, a fixing structure to maintain the cover and reservoir together and finally a support plate to receive the cassette and to adapt it into an electrophoresis box for usage. The fixing structure is preferably a liquid glue applied by lithography or silk-screen or a double-face tape, but could be any fixing structure that is not using heat for fixing and is therefore preventing the deformation of the cassette.

Because several technical difficulties occurred with traditional thermoforming method for the manufacture of the cassette, a thermoforming mold was prepared that allow homogenous plastic stretching and well-controlled process.

This mold requires a unique approach of construction. Stretching plastic is not a homogeneous and well-controlled process. To obtain the optimal results in the molding of the cassette of the present invention, the following are needed:
i) Male or female types of mold are selected based on the precision to obtain. Female type is used to produce pieces with larger details, while male molding provide more precision and allow molding pieces with smaller-narrower details;
ii) A groove is preferably carved around the mold to control the stretch of the plastic;
iii) Preferably, the mold is slightly elevated from the groove rear surface in order to stretch even more the plastic in the formed depression, which contribute to the symmetrical stretching of the plastic forming the cassette;
iv) A relative symmetry in the placing of the position stretch points around the molding cavities help to not misbalance-the stretching;
v) The use of vacuum holes in a sufficient quantity to generate an effective contact between the material surface and the groove rear surface of the mold and therefore a flat molding;
vi) Providing a chilling sufficient to cool rapidly the produced item, avoiding therein any deformation of the material surface;
vii) In the thermoforming machine and process, the heating of the plastic can be done with different types, size and strength of heating elements that will change the melting evenness of the plastic sheet prior thermoforming and allow a control of the plastic stretching during molding;
viii) Cooling elements can be introduced into the mold to affect and control the cooling evenness of the plastic during and after thermoforming process and prevent tension, twisting and deformation in the plastic due to wrong cooling or radiating heat;
ix) The residual heat coming from the surrounding plastic around the molded area is sufficient to radiate again into the molded part and to create bending, twisting or tensions, all deformation which will affect the molded part. It is preferable to minimize the radiating heat re-distribution during the molding process via die-cutting the molded part to detach it from the heated plastic roll. During this process, attachment points are usually left to allow manipulation of the molded part and easy removal from the machine. In the present invention, the minimum number and the smallest size of attachment points are preferred.

The drawings provided herewith are for the sole purpose of illustrating preferred embodiments of the invention, and shall not be considered as limiting the scope thereof.

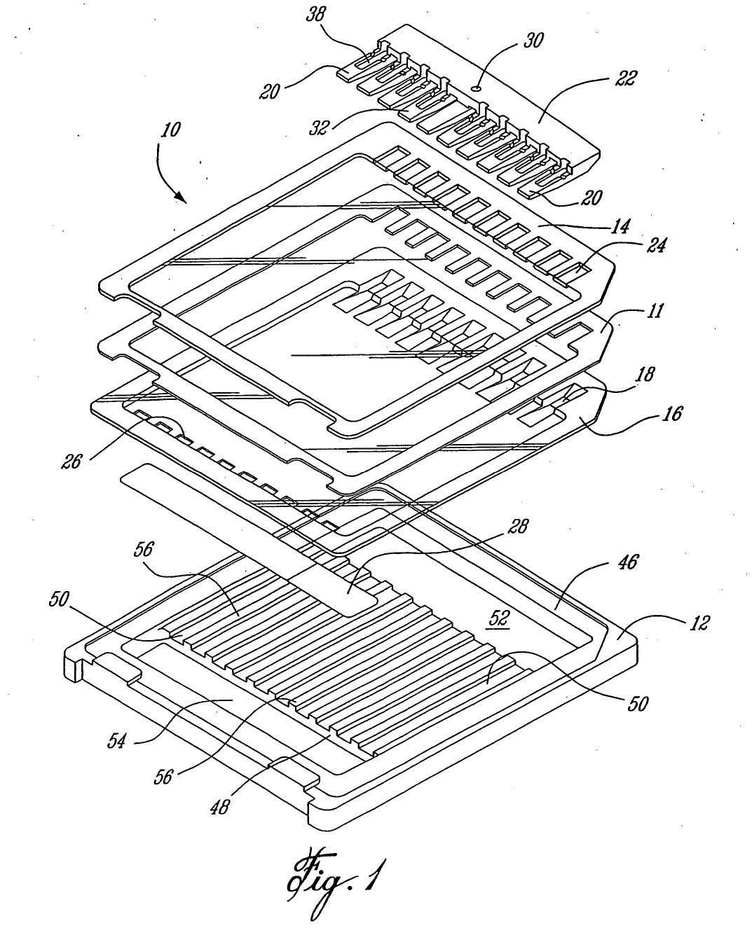

Referring to drawings, FIG. 1 illustrates a cassette assembly 10 and a support 12 therefor. Cassette 10 comprises a top plate 14 and a reservoir plate 16 each of a substantially square shape and having their four edges sealed, either with an adhesive layer 11 inserted therebetween, or with the help of any other compatible sealing means such as glue, ultrasonic welding, tape etc. The structure of layer 11 is complementary with that of both plates 14 and 16.

Plates 14 and 16 are preferably made of a chemically and electrically inert material and at least on of plates 14 and 16 is having the desired degree of rigidity to support and protect the gel during casting thereof, as well as shipping and handling operations. A thermoplastic “thermoformable” material is most preferred because the plates can be produced commercially via sheet thermoforming, which is quick, reliable and relatively cheap. Preferred thermoplastic materials suitable for the purposes of the invention include any electrically and chemically inert thermoplastic material that can be easily and economically thermoformed. Most preferred examples are polystyrene, high density polyethylene (HDPE), low density polyethylene (LDPE), linear low density polyethylene (LLDPE), polyethylene terephtalate (PET), glycol-modified PET, polyethylene naphthalate, polyvinyl chloride (PVC), polyvinylidene chloride (PVDC), polycarbonate, PMMA, Barex, Topas, polyvinylacetate (PVA), ethylene vinylacetate (EVA), polypropylene, polyesters, cellulose acetates, polyamides such as nylon™, and copolymers thereof. Preferably, both plates 14 and 16 are made of the same material for compatibility purposes. In addition, at least reservoir plate 16 should be transparent, but it is preferred that both plates 14 and 16 be transparent.

They could however be also made of a material suitable for “thin wall” injection molding as TPX, which has a lower density of about 0.9 that making it more fluid and allowing a more efficient injection of the plastic in the cavity to form a 20/1000 or more plastic wall. Any suitable materials for injection having a small density while heated are other desirable materials for the manufacture of the cassette.

Reservoir plate 16 comprises a series of reservoirs 18 for receiving a corresponding series of teeth 20 of comb 22. Top plate 14 has a complementary structure, i.e., a series of openings 24, that allow the passage therethrough of the plurality of teeth 20 for engagement into reservoirs 18. Reservoir plate 16 further comprises a series of slots 26 aligned with the series of reservoirs 18, and of substantially the same width. During filling, shipping and handling operations, these slots are sealed with a removable sealing strip 28 that will be removed before placing cassette 10 in the electrophoresis device. In an alternate embodiment, it has been found that the series of slots 26 can be replaced with slots having a smaller width but being present in a greater number, i.e, preferably twice the number of slots 26, with the same end result.

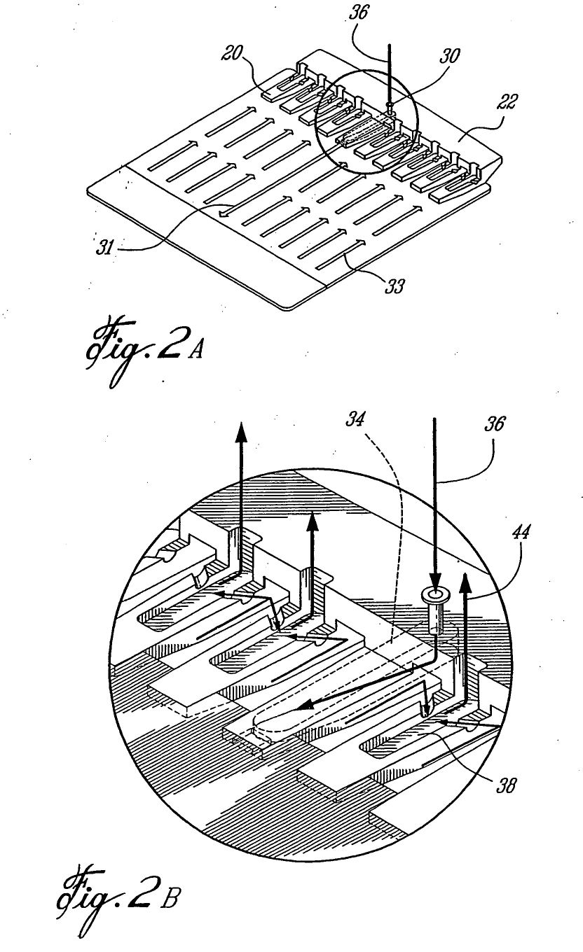

Comb 22 comprises an aperture or inlet 30 extending therethrough substantially perpendicularly to its longitudinal section, and aligned with a tooth 32, the latter comprising a longitudinal recess 34 shown in phantom lines in FIGS. 2A-2D and serving as an outlet. After engagement of teeth 20 into the series of reservoirs 18, an electrophoretic medium is injected into cassette 10 through aperture 30 and recess 34, as indicated by arrow 36. The flow of electrophoretic medium inside cassette 10 is also indicated by arrows 31 and 33. To ensure complete and proper filling of cassette 10, as well as minimizing air bubbles, a slight excess of electrophoretic medium must be injected. Such excess is discharged out of cassette 10 through a longitudinal recess 38 provided in each tooth 18. The flow of discharge is indicated by arrow 44. Recess 38 is located on the side of a tooth 20 that is opposite to the side of tooth 32 comprising recess 34. Each tooth 20 further comprises a pair of grooves 40 and 42, the depth of which being much smaller than that of recess 38, and arranged to form a V. The purpose of these grooves is mainly to facilitate gel separation from comb 22 upon removal thereof after completion of polymerization of the electrophoretic medium, although they may also be useful for discharge of excess of gel. Grooves 40 and 42 allow a clean separation of comb 22 from the gel, thus leaving a lower surface of reservoir 18 containing the medium substantially similar and even in each reservoir 18.

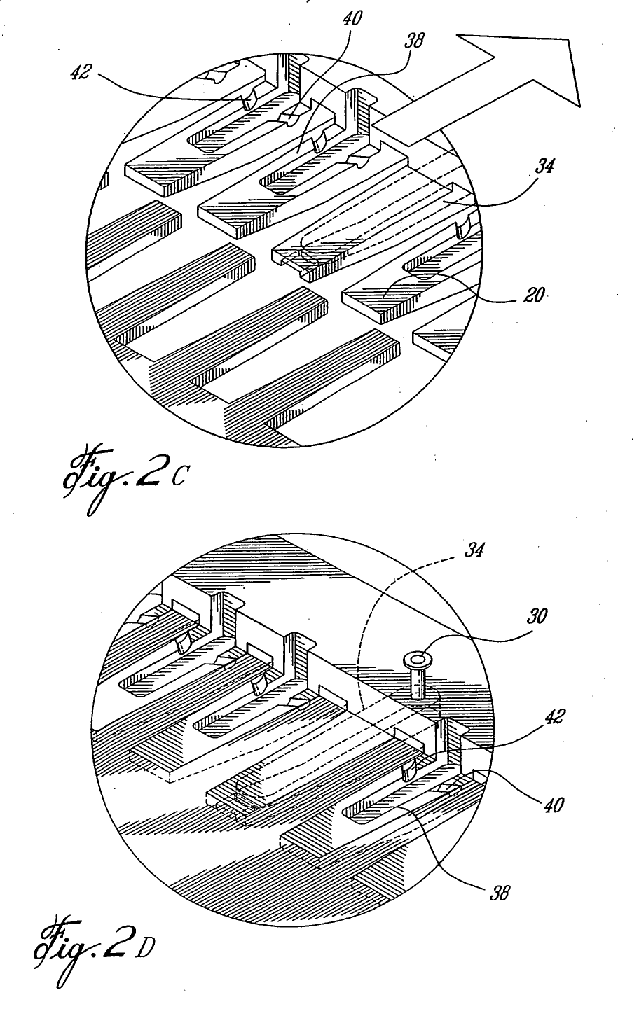

FIGS. 3A and 3B illustrate a preferred embodiment of the comb 22 of the present invention. Comb 22 comprises an aperture or inlet 30 extending therethrough substantially perpendicularly to its longitudinal section. After engagement of teeth 20 into the series of reservoirs 18, an electrophoretic medium is injected into cassette 10 through aperture 30. To ensure complete and proper filling of cassette 10, as well as minimizing air bubbles, a slight excess of electrophoretic medium must be injected. Each tooth 20 further comprises a protrusion 104 made of relatively soft and elastic material (such as rubber, urethane silicone, Chemraz, Viton, Buna-N, Aegis, Kalrez, Teflon, EPDM, Aflas, Neoprene, Fluorosilicone, Polyurethane and Mil-spec) and having the characteristic to both occupy and pressured the reservoir 18 and prevent liquid introduction between the teeth 20 and the cassette 10. When the protrusion 104 is made of a material known as an acrylamide polymerization inhibitor like silicone or urethane, it prevent acrylamide polymerization when the cassette 10 is filled with an electrophoresis medium.

During the electrophoretic medium casting process, the medium is poured into cassette 10 through opening 30 of comb 22, and allowed to solidify. Preferably, the cassette is held in a manner such that plates 14 and 16 are kept substantially parallel to facilitate the filling of the cassette. Plates 14 and 16 can be kept substantially parallel by, for example, applying a tension on each side thereof to stretch its position, or a “non-sticky” glue is applied on the external surface of the plates, so that the latter can be removably “stuck” during injection of the electrophoretic medium therebetween. Alternately, a vacuum can be applied both outside and inside the cassette, i.e., inside for drawing the gel inside the cassette, and outside for maintaining the plates substantially parallel. A combination of vacuum outside and positive pressure inside the cassette may also be used. The polymerization process begins after an excess of medium has poured out of each recess 38, confirming complete filling of cassette 10. This method therefore substantially eliminates air bubbles from cassette 10. Once polymerization is complete, cassette 10 is stored appropriately in a conventional manner.

Comb 22 is preferably removed only minutes prior to the use of the cassette, or immediately after complete polymerization of the gel, prior to storage, if the reservoirs 18 are well preserved from dehydration. At that point, it is slowly pulled out of the cassette, and each reservoir 18 is thereafter filled with an appropriate volume of a sample to be electrophoresed.

It is well known that in operation, the temperature of the electrophoresis gel increases. It is also well known that the temperature will be higher towards the middle of the cassette than on the sides thereof. As a result, the migration front of the products to be separated is altered, and erroneous interpretation might occur. A novel support plate has therefore been developed to overcome these problems, as well as for providing a proper profile maintenance, i.e., sufficient rigidity of the thin walls of the cassette, and facilitating installation of the cassette into an operational position in a conventional electrophoresis apparatus.

Support plate 12 comprises a frame 46 adapted to receive therein cassette 10, and comprising a surface 48 with a plurality of longitudinal recesses 50, which can be of any shape and size. Openings 52 and 54 are cut within the plate to define a free space substantially corresponding in size to reservoir 18 and slots 26. When cassette 10 is placed onto support 12, it lies directly onto ridges 56 of plate 12, thus forming a series of channels between recesses 50 and a surface of cassette 10 for circulation of the buffer solution therein (flow indicated by arrows 51), and thus helping dispersing heat generated within the cassette. As illustrated, each recess 50 is preferably aligned with a reservoir 18 and a slot 26, to ensure that the temperature of the migrating product and the gel is substantially the same, whether the reservoir is near the side or the middle of the cassette. It has however been found that such alignment is not mandatory. The critical element is that some buffer solution is allowed to circulate between the support plate and the cassette to “extract” heat from the latter. Support 12 can be made of any suitably rigid material, but is preferably made of a heat conducting material, so that heat is also extracted from ridges 56 that are in direct contact with the surface of cassette 10 lying thereon, and dispersed within the structure of the support. Cassette 10 can be maintained in place in plate 12 with the help of a couple of retainer plates 58.

With respect to the problem of interference of the polymerization process caused by the thermoplastic material of the cassette, it has been found that by combining a powerful initiator generating more free radicals with an appropriate “sticking” agent in the gel composition, there is no longer a need to apply a costly protective layer over the thermoplastic surfaces. Examples of such initiators include ammonium persulfate and N,N,N,N-tetramethylethylenediamine (TEMED); 4-dimethylaminopropionitrile; 1-hydroxycyclohexyl phenyl ketone; 2,2-diethoxy-acetophenone; 2,2-dimethoxy-2-phenylacetophenone; 2′,4′-dimethoxy-acetophenone; 2-hydroxy-2-methyl-1-propiophenone; 2-hydroxy-2-methyl-1-phenylpropan-1-one, and mixtures thereof. These strong initiators allow a substantially complete polymerization of the gel. However, the resulting polymerized gel does not stick to the plastic surface, which is critical, particularly in view of the fact that the cassette structure is relatively flexible. Detachment or unsticking of the polymerized electrophoretic medium from the cassette inner surfaces may lead to the introduction of undesirable air bubbles between the plastic surface and the gel, and may also cause irregularities in the medium structure, thus severely impairing the efficiency of the cassette. Surprisingly, it has been found that by adding to the gel composition a small amount of an adhesive compound is sufficient to allow the gel to adequately stick onto the plastic surface. The adhesive compound preferably corresponds to that used for coating the inner surfaces of currently available thermoplastic cassettes for the same purpose. However, the costs associated with the processing and coating of such a layer on the inner surfaces of the cassette are significant. On the other hand, in the present invention, all one has to do is to add a sufficient amount of the said adhesive compound into the gel composition to be injected into the cassette to achieve the same result. Not only is the procedure more simple, but the amount of adhesive compound required is smaller. Suitable adhesive compounds include polysilazanes or tetra-substituted silicon derivatives. The substituents can be the same or different, and include a straight or branched alkyl, alkoxy, ketone, ester or amide each comprising from 1 to 8 carbon atoms, or an amino, halogen, cyano or hydroxy. Preferred adhesives are alkyl alkoxy silane derivatives. Most preferred adhesives include Silane A-174, methacryloxytrimethoxysilylpropane, 3-(trimethoxysilyl)propyl methacrylate, 3-methacryloxypropyltrimethoxysilane, MEMO, DYNASYLAN MEMO, and γ-methacryloxypropyltrimethoxysilane.

The thickness of plates 14 and 16 should be sufficient to be rigid enough for operation in an electrophoresis system. For economical purposes, it has been found that it is not necessary to exceed a thickness of about 40/1000, preferably 20/1000.

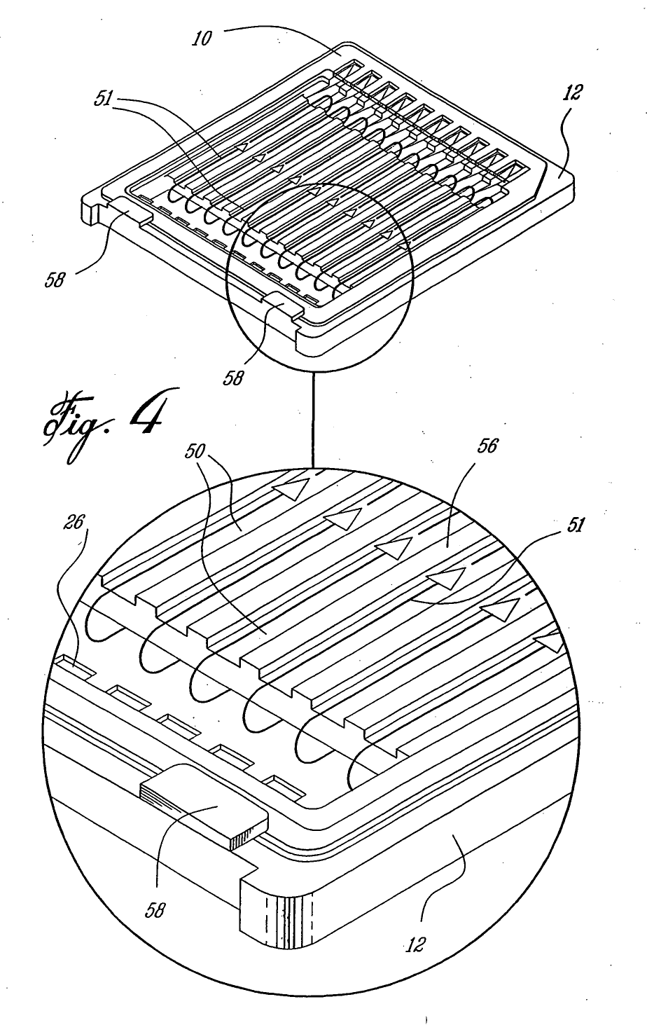

FIG. 5 is referred to a mold 80 comprising a frame 82, a vacuum outlet 84, a cooling inlet 86 and a cooling outlet 88. The frame 82 comprises a cavity 90 having a rear surface 92 perforated with a plurality of vacuum holes 94. Relatively centered to the rear surface 92 are a plurality of individual cassette molds 96 (either male or female molds), which are slightly elevated from the rear surface 92 to ensure a maximal stretch of the plastic during thermoforming. Each individual cassette mold 96 comprises pins 98 to assure a symmetrical stretching of the plastic during thermoforming. The individual cassette molds 96 each have a substantially square shape body defining an aperture 100 for the formation of a reservoir surface and a series of reservoir molding slots 102 above the aperture 100.

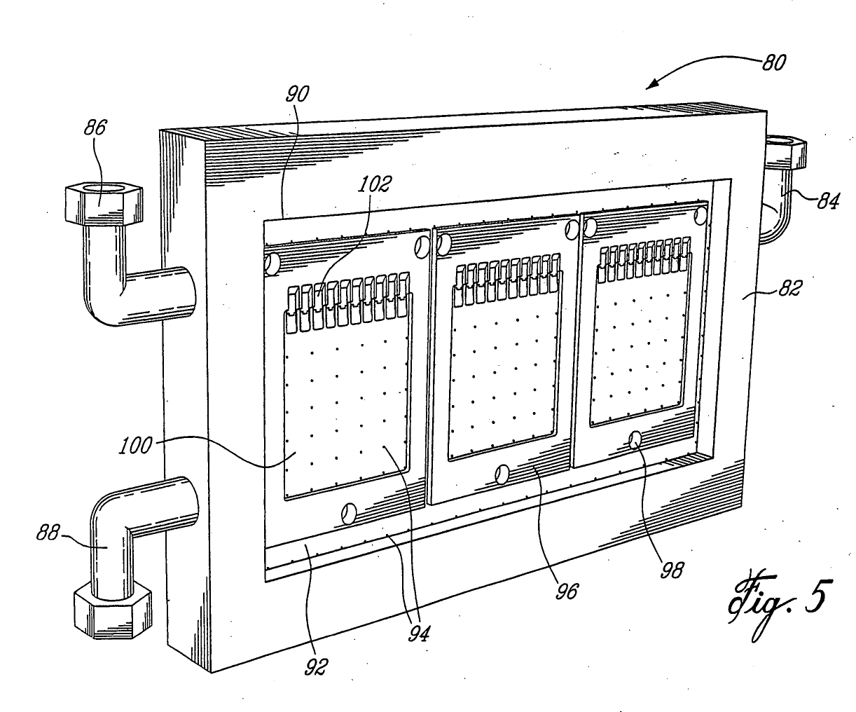

FIG. 6A is referred to the mold 80 comprising tubing 106 for conducting cooling or heating fluid through the mold 80. FIG. 6B represents another embodiment of the present invention, where the tubing 106 are installed in different zones of the mold 80, provided herein a different heating or cooling rate. In another embodiment of the present invention, heating elements differs for different zones in the mold 80, allowing a different heating to be performed for the different zones of the mold 80.

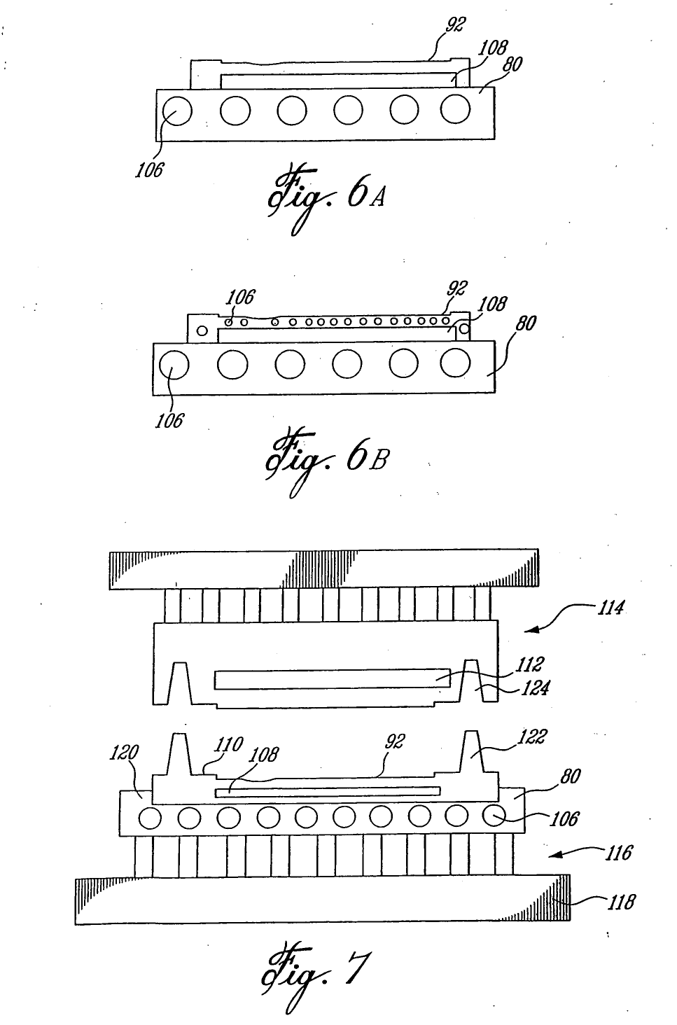

FIG. 7 is referred to another embodiment of the mold 80 comprising an upper part 114 and a lower part 116, the lower part 116 comprising a base 118 having a body 120 attached thereto. The body 120 having provided thereto heating/cooling elements 106, a vacuum chamber 108 to provide a sheet of material to be properly maintain in the mold 80 during molding, a cassette molding part 92 surrounded by a peripheral sheet engaging portion 110 and an engagement member 122. The upper part 114 is applied on the lower part 116 having the engagement member 122 adapting into a recess 124 and the upper part 114 providing a pressure to maintain a molded sheet material in a desired configuration while cooling. A pressure chamber 112 can provided in the upper part 114 to apply a pressure by compressed air on the molded sheet material in addition of the pressure from the upper part 114.

The electrophoresis cassette is composed of two distinct parts, a flat cover which is a non-thermoform thin PVC plate (it can also be replaced by any other material or thickness providing sufficient rigidity to the cassette) and a thermoformed thin piece. Both parts are assembled together preferably via a gluing process.

Production of the Cover (Silk-screen Printing)

The complete cassette is very sensitive to tension. The plastic could cause these tensions or the way the cassette being assembled. For instance, heating the plastic can release (desirable) or creates (undesirable) tensions depending under which conditions it is heated. Handling the plastic can also cause undesirable tensions. In order to manufacture cassette a reproducible way an approach which minimize such tensions was developed.

Print One Layer of Paint on Flatten PVC Sheets

The ink application has only decorative purposes.

Allow Complete Drying

The glue being a critical step in the product production, the paint needs to be properly cured to allow proper application and attachment of the glue. Commercial glues will be cured either via UV light exposition or air drying.

Print One Layer of Acrylic Based Glue on Painted Zone

The most common way to receive PVC is in large rolls. Under such format, the plastic keeps a memory of a curved shape following its position in the roll. This round shape is likely to create undesirable tensions in the final assembly. In order to prevent tension problems in the final cassette, there is a need to use flatten PVC sheets (flatten via slight heating and maintaining in a compressed area). Glue was also found the most appropriate approach to assemble the cassette. Since thin walls are used to constitute the electrophoresis cassette, the little amount of plastic present provide too little resistance and high thermal conductivity into the plastic, which can generate tension (such as curving and bending of the cassette), so classic welding approaches to attach these plastic pieces together are not acceptable and had to be replaced by gluing. Gluing also present an advantage to make the cassettes easy to open after electrophoresis. The simplest way to apply the glue is through silk-screen printing (screen printing, silk screening engraving) or even application of double face tape.

The choice of glue is critical since several glues are likely to affect the electrophoresis pattern when the gel is in use. Acrylic based glue with minimal amounts of organic or inorganic solvent are preferred. Such types of glue are commercially available are need to be either cured using UV light exposition, air drying or direct.

Die-cut Wells' Holes and Contour

Die cutting of the plastic piece is required to allow final assembly. Normal and classic blade based die can be used. Water knifes of laser cutter can also be used for this process.

Production of the Reservoir

Preferably, the production of the reservoir is performed using thermoforming. However, it is also contemplate to use a “thin wall” injection technique to arrive to another embodiment of the present invention.

Due to the usage of thin plastic, this step requires a proper control of the molding process in order to prevent any tension, curving, bending or deformation in the thermoformed part. The clarity, transparency of the plastic must also be preserved through the process.

Installation of the Material Sheet on Production Mold and Thermoforming Machine

Preferably 20/1000 thick plastic in a roll form or in sheet can be used as based material for production. Thinner or thicker plastic can also be used, but better results based on structural qualities and plastic waste is obtained with such material.

Produce Thermoformed Pieces

The thermoforming process can be done from sheets of plastic, one sheet at a time, however, this process is preferably made in continuous, a plastic roll passing through a heating system, followed by a pressing and thermoforming station, followed by a die-cutting station, which can be also done simultaneously with the thermoforming, followed by a thermoformed pieces removing station.

With all thermoforming, dry sheet is heated to a controlled softening temperature, stretched to conform to the mold contours and cooled to the temperature at which the part becomes rigid and maintains the desired shape. The formed part is trimmed to eliminate edges and fabricated into the final configuration.

Thermoforming can be performed on any thermoforming machine; however, key parameters must be respected. For instance, heating is preferably made by radiating heat (keep transparency). Standard heating system is preferably used; however, more controllable results could be used using different heating elements with different heating strength. Once properly heated the plastic sheet is displaced over the thermoforming area to be shaped. Process using vacuum forming (using a negative pressure between the sheet and the mold), or pressure forming (using a positive pressure on the opposite side of the sheet and a negative pressure between the sheet and the mold can be used with success. Due to the high specification and flatness, evenness requirements, a rapid and strong cooling is preferably used. The mold base is constituted of tubing and channels to allow coolant liquids like water or other cooling material. In the actual case, we force cooling by using pre-chilled water to pass through the mold. The channels in the mold can be designed to offer different level and speed of cooling in order to control the stretch and cooling performance in specific and well define areas of the mold. In the preferred approach, the cooled piece is immediately die-cut to be separated from the rest of the hot plastic sheet and then to prevent heat radiation to return into the molded part as it leaves the thermoforming area.

Produce by Strips of 3 Cassettes to Use Full Width of Plastic Roll.

Since it is imperative to keep the molded part away from any deformations (due to manipulation or heat radiation), the minimal residual plastic is left around the molded part. The maximum of a standard size plastic roll is used; the thermoforming area will then occupy most of the width of the plastic roll, minimizing lost and warm plastic that could radiate back.

Die-cut Cassette Bottom Holes

In order to allow contact between the polyacrylamide gel and the buffer and the electrode, holes need to be made at the bottom of the cassette. A punch is preferably used to perform the holes in order to minimize mechanical tension due to pressure apply by knifes or blades on the plastic sheets.

Assemble Both Part Under Press

The thermoformed 3 cavity strips are cleaned and installed on a jig. The silk-screen produced cover sheet with its 3 glued area matching the 3 cassette thermoformed cavities area also positioned on the jig to allow binding via pressure.

Die-cut Cassette Out of Assembled Piece

The assembled strips are then die-cut using a standard blade based die to liberate 2 cassettes ready to be filled.

On top of producing the cassettes, filling them with the gel solution is also requiring an inventive manufacturing process. The electrophoresis cassettes being made of thin and relatively soft plastic sheets, they need to be positioned in a form (or exoskeleton) to shape their final configuration until the gel is entirely polymerized. Filling can be done in the exoskeleton chamber with or without its comb and the gel poured in the cassette via the top opening or via a hole in the comb until the exact quantity or level is attained. After complete polymerization, the cassette is removed from the exoskeleton to be bagged or used.

As an expandable small to large-scale production method, a preferred filling process is:

The bottom holes of the cassette are blocked by a plastic or rubber tape (like electric tape) to prevent liquid acrylamide leakage.

The cassette is maintained in a vertical position in a fixture of some sort. Such fixture should not affect the plastic flexibility in front or back sides of the gel area.

A known amount (example 6.4 ml for an 8×8×1 cm cassette reservoir) of polyacrylamide solution is poured in the reservoir area.

The comb (plastic or hybrid) is positioned on the top of the gel in the well cavity area. At this stage, due to flexibility of the plastic sheets, the liquid acrylamide generate a pressure on the cassette walls having them behave as a balloon. The level of solution is much lower than the comb position. Such level could not allow proper electrophoretic separation.

The filled cassette is delicately moved into exoskeleton. This fixture is then delicately closed to press in sandwich the cassette between the two solid walls of the fixture and give to the soft cassette the shape it should have in order to generate an adequate flat and even electrophoretic separation area in the polyacrylamide gel.

The gels can be polymerized with this method either by chemical stimulation using ammonium persulfate and TEMED, or via UV polymerization using initiators like 1-hydroxycyclohexyl phenyl ketone, 2,2-Diethoxyacetophenone, 2,2 Dimethoxy-2-phenylacetophenone, 2′,4′-Dimethoxyacetophenone, 2-Hydroxy-2-methyl-1-propiophenone, 2-Hydroxy-2-methyl-1-phenyl-propan-1-one.

Gradient gels (example 4% to 20% acrylamide and other recipes) as well as continuous gels can be produced using this approach.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A mold for the manufacture of an electrophoresis cassette, said mold comprising a body having a cassette molding part formed on one face thereof, said cassette molding part being surrounded by a peripheral sheet engaging portion extending in a plane located at a different elevation than said cassette molding part to provide for substantially uniform stretching of the sheet on the cassette molding part.

2. The mold of claim 1, further comprising:
a vacuum outlet provided on the body; and
a plurality of vacuum apertures defined in said body.

3. The mold of claim 1, further comprising:
a cooling inlet provided on the body; and
a cooling outlet provided on the body.

4. The mold of claim 1, wherein said at least one cassette mold is a male cassette mold and wherein said peripheral sheet engaging portion is a groove provided in said body.

5. The mold of claim 1, wherein said at least one cassette mold is a female cassette mold and wherein said peripheral sheet engaging portion is a shoulder provided in said body.

6. The mold of claim 1, wherein said at least one cassette mold comprises at least two relatively symmetrical stretching members provided at the upper and lower extremities of said cassette mold for stretching said material.

7. The mold of claim 2, wherein said vacuum outlet is secured on one side of the mold.

8. The mold of claim 3, wherein said cooling inlet is secured on one side of the mold.

9. The mold of claim 3, wherein said cooling outlet is secured on one side of the mold.

10. A thermoforming mold for manufacturing an electrophoresis cassette, the mold comprising:
a body having a cassette molding part formed on one face thereof, said cassette molding part being surrounded by a peripheral sheet engaging portion extending in a plane located at a different elevation than said cassette molding part to provide for substantially uniform stretching of the heated sheet on the cassette molding part.

11. The thermoforming mold of claim 10, further comprises a plurality of vacuum holes defined in said body to provide assistance in holding the heated sheet in a flat state on said body, and a vacuum port for allowing said body to be connected to a vacuum source.

12. A molding method for the manufacture of an electrophoresis cassette comprising the steps of:
a) heating a thermoforming material applied on a thermoforming mold suitable for the manufacture of an electrophoresis cassette;
b) applying a pressure on said material to closely maintain the material on said mold;
c) stretching said material to obtain an uniformly distributed material surface;
d) cooling said material to form a molded material surface with cooling parameters adapted to provide an uniformly distributed material surface; and
e) providing holes in a peripheral portion of the electrophoresis cassette facing a peripheral portion comprising reservoirs entries.

13. The molding method of claim 12, wherein said mold is comprising a frame having a groove provided in one face thereof and having a groove rear surface, said groove being of dimensions sufficient to have at least one cassette mold provided therein while keeping a space between an outline of the at least one cassette mold and an inside border of the groove to permit stretching of a sheet material in a uniform molded material surface.

14. The molding method of claim 12, wherein heating said thermoforming material is performed before applying said material on the mold.

15. The molding method of claim 12, wherein said mold further comprises a vacuum outlet provided on the frame and a plurality of vacuum apertures defined in said frame, and wherein the step of applying pressure is performed by at least in part drawing air through said vacuum apertures so as to create a negative pressure between said cassette mold and the sheet material.

16. The molding method of claim 12, wherein said mold is divided in a plurality of zones and wherein said heating is performed at different temperature and/or during heating time periods for said zones.

17. The molding method of claim 12, wherein said heating is performed by radiating heat from said mold.

18. The molding method of claim 12, wherein said cooling step is performed by passing a cooling fluid through cooling channels provided in said mold.

19. The molding method of claim 12, wherein said cooling step comprises the pre-cooling of said fluid passing through cooling channels provided in said mold.

20. The molding method of claim 12, wherein said mold is divided in a plurality of zones and wherein said cooling is performed at different speed for each zone.

21. The molding method of claim 12, wherein a minimal amount of the material is left around the mold for minimizing heat propagation from left material to molded material.

22. The molding method of claim 12, wherein said holes are punched in said cassette to minimize mechanical tensions created in said cassette.

23. A method for filing an electrophoresis medium into an electrophoresis cassette comprising the steps of:
a) sealing at least one aperture of said cassette;
b) injecting the electrophoresis medium into said cassette;
c) applying a pressure onto said cassette in order to generate a electrophoresis separation area shaped in said electrophoresis medium in a form adapted to electrophoresis.

24. The method of claim 23, wherein said injecting is performed by injecting the electrophoresis through an aperture in a comb inserted in said cassette.

25. The method of claim 23, further comprising the step of inserting a comb into said cassette to form well cavity after step b)

26. A comb for an electrophoresis cassette adapted to be removably inserted into said cassette comprising at least one tooth having protrusion provided thereto for preventing the electrophoresis medium gel attachment to said comb.

27. The comb of claim 26, further allowing injection therein of an electrophoresis medium and comprising at least one inlet for injection of the electrophoresis medium.

28. The comb of claim 26, wherein said at least one tooth is made of hard plastic or polymer.

29. The comb of claim 26, wherein said at least one protrusion is made of a material selected from the group consisting of rubber, urethane silicone, Chemraz, Viton, Buna-N, Aegis, Kalrez, Teflon, EPDM, Aflas, Neoprene, Fluorosilicone, Polyurethane and Mil-spec.

30. A comb for an electrophoresis cassette adapted to be removably inserted into said cassette comprising at least one tooth having protrusion provided thereto for preventing acrylamide polymerization surrounding said comb.

31. The comb of claim 30, further allowing injection therein of an electrophoresis medium and comprising at least one inlet for injection of the electrophoresis medium.

32. The comb of claim 30, wherein said at least one tooth is made of hard plastic or polymer.

33. The comb of claim 30, wherein said at least one protrusion is made of a material inhibiting acrylamide polymerization.

34. The comb of claim 30, wherein said at least one protrusion is made of a material selected from the group consisting of rubber, urethane silicone, Chemraz, Viton, Buna-N, Aegis, Kalrez, Teflon, EPDM, Aflas, Neoprene, Fluorosilicone, Polyurethane and Mil-spec.
